(19) United States
(12) Patent Application Publication
(10) Pub. No.: US 2024/0294466 A1
(43) Pub. Date: Sep. 5, 2024

(54) SODIUM N-(8-(2- HYDROXYBENZOYL)AMINO)CAPRYLATE POLYMORPHIC FORM A

(71) Applicant: Novo Nordisk A/S, Bagsvaerd (DK)

(72) Inventors: Thomas Kvistgaard Vilhelmsen, Solroed Strand (DK); Zacharias Brimnes Visby Damholt, Bagsvaerd (DK)

(21) Appl. No.: 18/578,485

(22) PCT Filed: Jul. 14, 2022
(86) PCT No.: PCT/EP2022/069705

§ 371 Date: Jan. 11, 2024

(30) Foreign Application Priority Data
Jul. 16, 2021 (EP) 21186179.4

(51) Int. Cl. C07C 235/60; A61K 9/16; A61K 9/20

(52) U.S. Cl. CPC C07C 235/60; A61K 9/1617; A61K 9/2013; C07B 2200/13

(57) ABSTRACT

The present invention relates to a method of making sodium N-(8-2-Hydroxybenzoyl)amino capry late form A. SNAC polymorphic form A having improved stability and the use of said SNAC polymorphic form A in a solid pharmaceutical dosage form

The present invention relates to a method of making sodium N-(8-2-Hydroxybenzoyl)amino caprylate form A, SNAC polymorphic form A having improved stability and the use of said SNAC polymorphic form A in a solid pharmaceutical dosage form.

### TECHNICAL FIELD

There is a significant pharmaceutical need to improve the oral bioavailability of many active compounds. There are many factors that are inherent either to the active compound or to the absorptive interface that limits a substance's rate and extent of absorption after oral intake.

Pharmaceutical excipients are inactive substances other than the active pharmaceutical ingredient (API), which are included in a drug formulation to serve several purposes. Pharmaceutical excipients can modify drug absorption, pharmacokinetics, and drug stability, and may also help to overcome limitations of the API in terms of manufacturability.

For instance, oral administration of therapeutic peptides is hindered by poor absorption across the gastrointestinal barrier and extensive degradation by proteolytic enzymes. Absorption enhancers can promote membrane permeability and improve oral bioavailability. Sodium N-[8-(2-hydroxybenzoyl)amino]caprylate (SNAC) is an example of such an absorption enhancer that has good safety and has been reported to enhance the permeability of a diverse spectrum of molecules including peptides such as semaglutide (e.g. WO 2012/080471) and proteins, such as insulin (Abbas et al., 2002), calcitonin (Buclin et al., 2002) and other macromolecules such as heparin (unfractionated heparin and two different low-molecular weight heparins) (Brayden et al., 1997, Leone-Bay et al., 1998a, Leone-Bay et al., 1998b, Money, 2001, Pineo et al., 2001). General preparation protocols of SNAC are set out in WO 2000/46182 and WO 2000/59863. WO 2008/028859 describes improved methods for the synthesis of N-(8-[2-hydroxybenzoyl]-amino) caprylic acid and its sodium salts.

The components of pharmaceutical solid dosage forms must be stable under various environmental conditions, during production and in the final medicinal composition such as the packed drug product so that stability during long-term storage can be guaranteed. It is desirable that the components of a pharmaceutical dosage composition display low hygroscopicity. Typically, pharmaceutical dosage composition not displaying low hygroscopicity and sufficient stability are not optimal for being handled at an industrial scale as they would require low relative humidity conditions and/or temperature settings below room temperature while manufacturing and it might be limiting for the scale of manufacturing as well. The insufficient stability might also result in refrigerated or even frozen storage requirements for the pharmaceutical dosage composition and might increase demands for expensive moisture tight packaging systems. The term stability of a pharmaceutical solid dosage such as the final packed drug product form implies the physical and chemical integrity of the API, the excipients, and intactness of packaging. Any unintended change in the inherent nature and physicochemical characteristics of pharmaceutical excipients could lead to potential instabilities in the formulation that could disrupt the quality and performance attributes of the product. For instance, physical instability may involve phase transformation of the excipients, which may be due to e.g. polymorphic changes, hydration and dehydration, precipitation, or changes in the amorphous or crystalline nature. Polymorphism is a well-established phenomenon which describes the ability of a solid-state molecular structure to be repetitively positioned in at least two different arrangements in three-dimensional space. These different arrangements can result in different sets of physicochemical properties of the same molecular structure, which can significantly affect material behaviour during handling, processing, and storing. Put differently, differences in polymeric forms could, in some cases, affect the quality or performance of a drug product. For further details, see ICH guideline Q6A. Consequently, polymorphism must be taken into consideration during every processing stage starting from early steps such as preformulation and formulation development, passing through processing, manufacturing, and storage, and eventually until consumption in humans.

As described in WO 2005/107462, SNAC crystallises in several different polymorphic forms, each of which having specific properties.

Given the favourable properties of SNAC with respect to increasing bioavailability of orally delivered biologics such as peptides and proteins, there is still a strong need for the development of methods of producing SNAC in its desired polymorphic form displaying low hygroscopicity in an effective way.

### SUMMARY OF THE INVENTION

The invention relates in a first aspect to a process of reducing the hygroscopicity of monosodium N-[8-(2-hydroxybenzoyl)-amino]caprylate (SNAC) form A, the process comprising the following steps:
a. providing SNAC polymorphic form A;
b. heating, optionally under reduced pressure, the SNAC polymorphic form A provided in step a. at a temperature of above 90° C., such as at a temperature about 105-140° C. for at least about 5 minutes, such as at least about 15 minutes.

In some embodiments, there is provided a method of producing monosodium N-[8-(2-hydroxybenzoyl)-amino]caprylate form A, the method comprising the steps of
a. suspending or dissolving N-[8-(2-hydroxybenzoyl)-amino]caprylic acid in suitable solvent such as isopropanol;
b. adding a molar excess of a sodium containing salt such as sodium hydroxide as an aqueous solution to form monosodium N-[8-(2-hydroxybenzoyl)-amino]caprylate;
c. isolating the so-formed monosodium N-[8-(2-hydroxybenzoyl)-amino]caprylate;
d. heating, optionally under reduced pressure, the monosodium N-[8-(2-hydroxybenzoyl)-amino]caprylate at a temperature of above 90° C., such as at a temperature of about 105-140° C., for at least about 5 minutes, such as for at least about 15 minutes.

The invention relates in a second aspect to SNAC polymorphic form A wherein said SNAC polymorphic form A is characterised by exhibiting a mass increase of 1.5% or less when subjected to an increase in relative humidity from about 0% to about 65% relative humidity (RH) at 25° C. as determined by DVS and/or wherein the peak at angles of diffraction 2Theta (2θ) of 8.7±0.2° measured using CuKa radiation has a FWHM of below 0.9° (2θ).

The invention relates in another aspect to SNAC polymorphic form A obtainable by a process according to the first or the alternative first aspect.

The invention in a third aspect relates to the use of SNAC polymorphic form A obtainable by a process according to the first or alternative first aspect for the manufacture of a SNAC granule and/or a solid dosage form. Also, or alternatively a third aspect relates to the use of SNAC polymorphic form A according to the second or alternative second aspect for the manufacture of a SNAC granule and/or a solid dosage form

In a fourth aspect, the invention relates to a solid pharmaceutical composition comprising SNAC polymorphic form A according to the second or alternative second aspect of the invention. In some embodiments, the SNAC polymorphic form A may be in form of granules. In some embodiments, the SNAC polymorphic form A may be in form of a powder. In some embodiments, the SNAC polymorphic form A may be in form of particles.

### BRIEF DESCRIPTION OF DRAWINGS

FIG. 1 shows an X-ray Powder Diffraction “XRPD” pattern of SNAC polymorphic form A exhibiting a mass increase of less than 1.0% when subjected to an increase in relative humidity from about 0% to about 65% RH at 25° C. as determined by DVS

FIG. 2 shows an XRPD pattern of SNAC polymorphic form A exhibiting a mass increase of about 1.3% when subjected to an increase in relative humidity from about 0% to about 65% RH at 25° C. as determined by DVS.

FIG. 3 shows an XRPD pattern of SNAC polymorphic form A exhibiting a mass increase of about 2.9% when subjected to an increase in relative humidity from about 0% to about 65% RH at 25° C. as determined by DVS.

FIG. 4 shows an XRPD pattern of SNAC polymorphic form E.

FIG. 5 shows an XRPD pattern of a mixture of polymorphic form A and E.

FIG. 6 shows an XRPD pattern of SNAC polymorphic form F.

FIG. 7 shows an XRPD pattern of a mixture of SNAC polymorphic form A and F.

FIG. 8 shows an XRPD pattern of SNAC polymorphic form B.

FIG. 9 shows an XRPD pattern of a mixture of SNAC polymorphic form A and B.

FIG. 10 shows an XRPD pattern of amorphous SNAC.

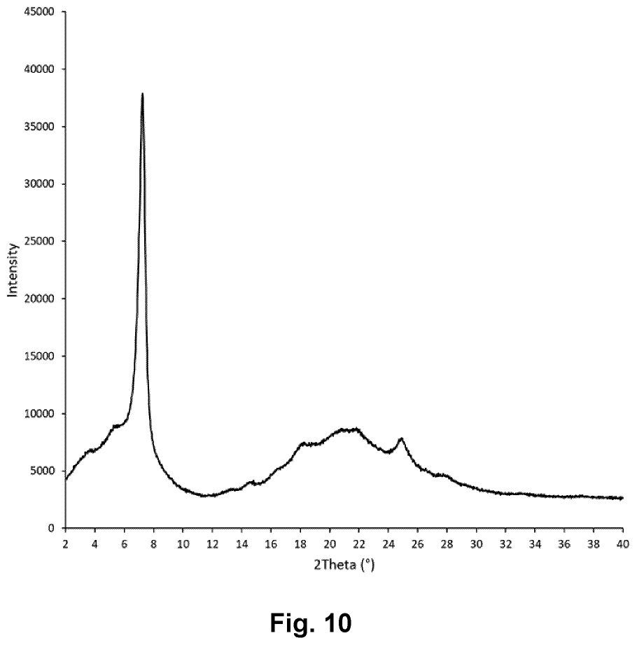

FIG. 11 exemplifies how the FWHM (° 2θ) for the XRPD peak 8.7±0.2° (2θ) was calculated using auto mode baseline.

FIG. 12 exemplifies how the FWHM (° 2θ) for the XRPD peak 8.7±0.2° (2θ) was calculated using manual mode baseline.

FIG. 13 shows DVS isotherm plots of SNAC polymorphic form A with different hygroscopicity.

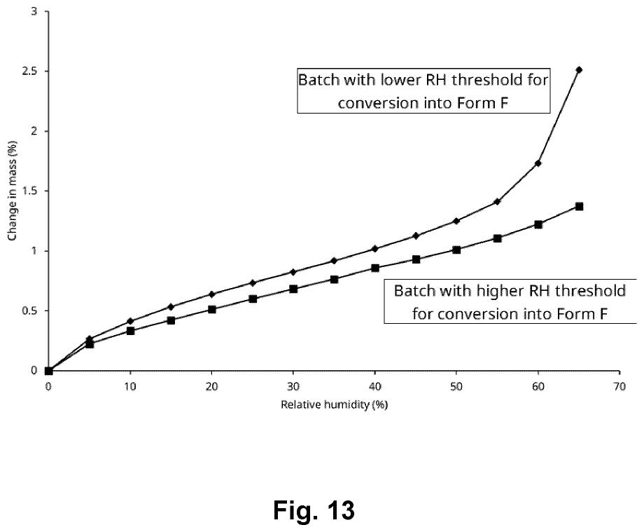

### DESCRIPTION

SNAC polymorphic form A may be prepared according to Example 2 of WO 2008/028859. SNAC polymorphic form A exhibits an X-ray powder diffraction pattern comprising peaks at angles of diffraction 2Theta (2θ) of 3.0±0.2°, 6.0±0.2, 8.7±0.2°, 11.6±0.2°, 14.6±0.2°, and 18.9±0.2° measured using CuKa radiation. As described in WO 2008/028859, N-[8-(2-hydroxybenzoyl)-amino]caprylic acid is reacted with a molar excess of sodium salt to form SNAC. As the reaction is carried out in an aqueous solvent a trihydrate form of SNAC is formed and then converted into form A by drying under reduced pressure. The drying step may be carried out in an oven under vacuum as described in WO 2008/028859. The drying step may be carried out in an oven under vacuum as described in WO 2005/107462. However, such a drying method may be found insufficient for bulk drying a large quantity of material, such as the quantity typically used for an industrial scale production, in an efficient manner. An industrial scale production of SNAC typically is the range of up to a few tons. Usually vacuum dryers that can rotate and/or stir are required for pursuing an industrial scale manufacturing process. Non-limiting example of suitable dryers are conical, biconical, spherical, paddle dryers, and tray dryer.

During efforts of up-scaling the process for producing SNAC polymorphic form A, the present inventors have observed notable differences in the quality of SNAC polymorphic form A. For instance, it was noted that certain batches of SNAC polymorphic form A were more hygroscopic than others. The inventors also observed that storage stability of SNAC polymorphic form A differed from batch to batch and may be related to the hygroscopic differences. After careful analysis, the corresponding XRPD pattern pointed to the presence of varying degrees of crystal imperfections in the SNAC polymorphic form A that explained the increased hygroscopicity.

During testing and development of an up-scaled process, the inventors have surprisingly found that heating SNAC polymorphic form A at a temperature of above 90° C., such as at a temperature about 105-140° C. for at least about 5 minutes, such as for at least about 15 minutes results in a notable reduction in crystal imperfections. Reducing crystal imperfections in SNAC polymorphic form A results in a significant decrease in hygroscopicity of SNAC polymorphic form A and therefore an improved storage stability. This enables a more efficient use of SNAC polymorphic form A in the manufacturing of pharmaceutical solid dosage forms because the relative humidity and temperature during manufacturing do not need to be low and might even enable manufacturing under ambient conditions. Furthermore, the improved SNAC polymorphic form A enables the use of packaging systems that are permeable to moisture. An advantage of such packaging is that it is simpler and cheaper, while still allowing for an acceptable or even prolonged shelf-life, which consecutively improves the convenience for the user.

In a first aspect the invention relates to a process of reducing the hygroscopicity of monosodium N-[8-(2-hydroxybenzoyl)-amino]caprylate (SNAC) form A, the process comprising the following steps:
A. providing SNAC polymorphic form A;
B. heating, optionally under reduced pressure, the SNAC polymorphic form A provided in step A. at a temperature of above 90° C., such as at a temperature of about 105-140° C. for at least about 5 minutes, such as for at least about 15 minutes.

In some embodiments, SNAC polymorphic form A provided in step A, exhibits a mass increase of more than 1.5% when subjected to an increase in relative humidity from about 0% to about 65% relative humidity (RH) at 25° C. as determined by DVS

In some embodiments SNAC polymorphic form A has an X-ray powder diffraction pattern comprising peaks at 3.0°, 6.0°, 8.7°, 11.6°, 14.6°, and 18.9° (20±0.2°) such as 2.9°, 5.8°, 8.6°, 11.4°, 14.4°, and 18.8° (20+)0.1°, when measured using Cu Ka radiation. In some embodiments SNAC polymorphic form A has an X-ray powder diffraction pattern comprising peaks at 2.94±0.06°, 5.82±0.05°, 8.55±0.08°, 11.45±0.15°, 14.4±0.2°, and 18.87±0.08°, when measured using Cu Ka radiation.

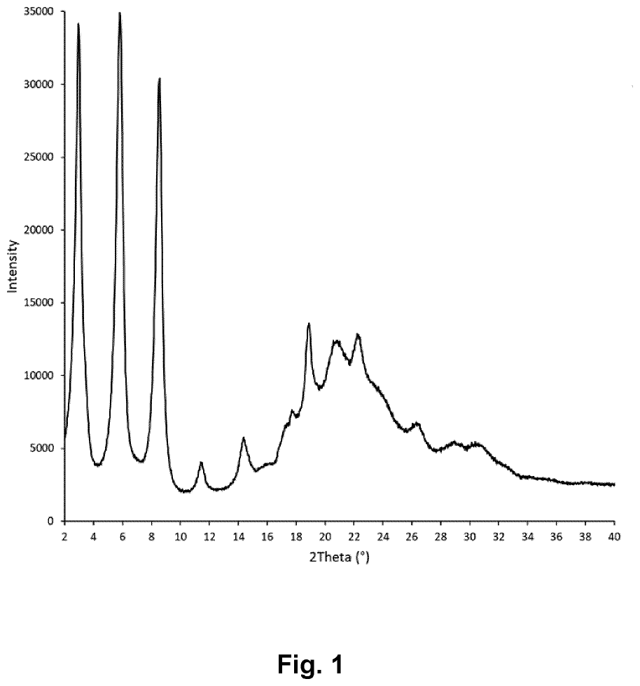

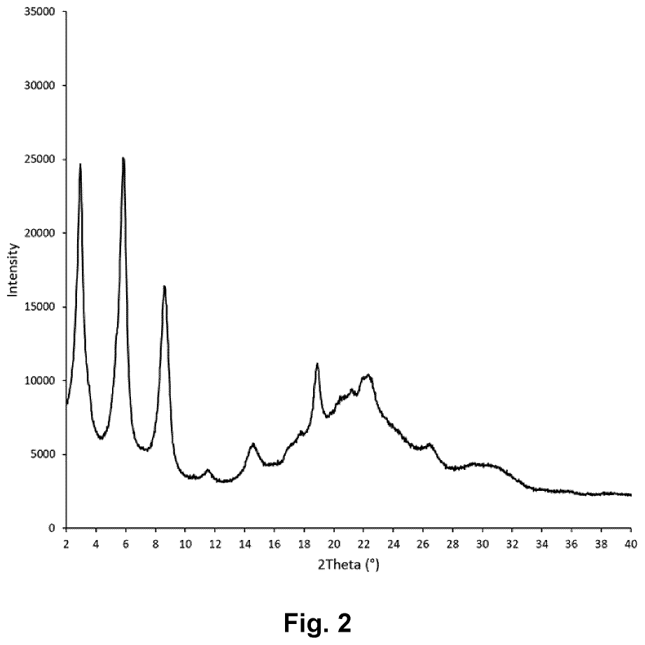

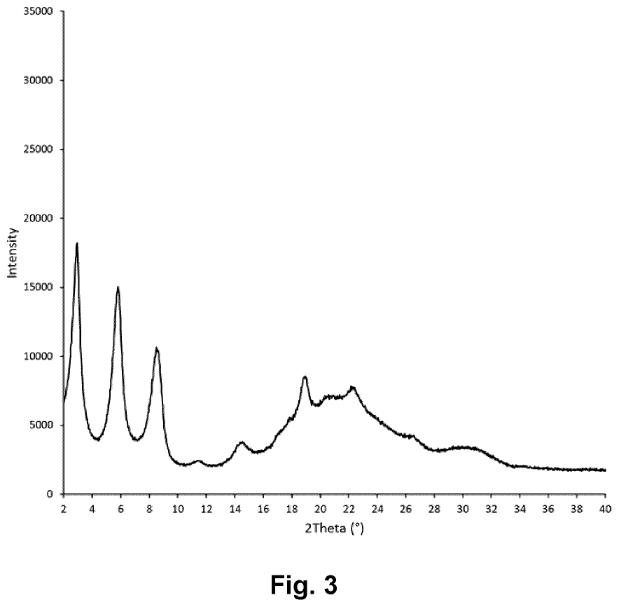

In some embodiments SNAC polymorphic form A according to the invention has a representative X-ray powder diffraction pattern as provided in FIG. 1 or 2. In some embodiments SNAC polymorphic form A provided in step A has a representative X-ray powder diffraction pattern as provided in FIG. 3.

In some embodiments heating is carried out at a temperature of about 105-140° C., such as at a temperature of about 105-135° C.

In some embodiments heating is carried out at a temperature of about 110-140° C., such as at a temperature of about 110-135° C., such as at a temperature of about 111-130° C. In some embodiments the heating is carried out at a temperature of about 115-124° C., such as at about 120° C. In some embodiments the heating is carried out at a temperature of about 111° C. or at a temperature of about 112° C. or at a temperature of about 113° C. or at a temperature of about 114° C. or at a temperature of about 115° C. or at a temperature of about 116° C. or at a temperature of about 117° C. or at a temperature of about 118° C. or at a temperature of about 119° C. or at a temperature of about 120° C. or at a temperature of about 121° C. or at a temperature of about 122° C. or at a temperature of about 123° C.

In some embodiments heating is carried out at a temperature of about 105-114° C., such as at about 110° C.

In some embodiments the heating is carried out at a temperature of about 125-134° C., such as at about 130° C.

In some embodiments the heating is carried out for at least 20 minutes or at least 30 minutes or at least 45 minutes or at least 1 hour or at least 2 hours or at least 3 hours or at least 4 hours or at least 5 hours or at least 6 hours or at least 7 hours or at least 8 hours or at least 9 hours or at least 10 hours or at least 11 hours or at least 12 hours or at least 18 hours or at least 24 hours.

In some embodiments the heating is carried out for less than 72 hours such as less than 66 hours, such as less than 60 hours such as less than 54 hours such as less than 48 hours such as less than 42 hours such as less than 36 hours such as less than 30 hours.

In some embodiments the heating is carried out between about 15 minutes and 24 hours. In some embodiments the heating is carried out between about 20 minutes and 24 hours. In some embodiments the heating is carried out between about 30 minutes and 24 hours. In some embodiments the heating is carried out between about 45 minutes and 24 hours. In some embodiments the heating is carried out between about 1-24 hours. In some embodiments the heating is carried out between about 6-24 hours.

In some embodiments the heating is carried out between about 1-72 hours. In some embodiments the heating is carried out between about 6-72 hours. In some embodiments the heating is carried out between about 12-72 hours. In some embodiments the heating is carried out between about 18-72 hours. In some embodiments the heating is carried out between about 24-72 hours.

In some embodiments the heating is carried out between about 1-60 hours. In some embodiments the heating is carried out between about 6-60 hours. In some embodiments the heating is carried out between about 12-60 hours. In some embodiments the heating is carried out between about 18-60 hours. In some embodiments the heating is carried out between about 24-60 hours.

In some embodiments the heating is carried out between about 1-54 hours. In some embodiments the heating is carried out between about 6-54 hours. In some embodiments the heating is carried out between about 12-54 hours. In some embodiments the heating is carried out between about 18-54 hours. In some embodiments the heating is carried out between about 24-54 hours.

In some embodiments the heating is carried out between about 1-48 hours. In some embodiments the heating is carried out between about 6-48 hours. In some embodiments the heating is carried out between about 12-48 hours. In some embodiments the heating is carried out between about 18-48 hours. In some embodiments the heating is carried out between about 24-48 hours.

In some embodiments the heating is carried out between about 0.5-36 hours. In some embodiments the heating is carried out between about 1-36 hours. In some embodiments the heating is carried out between about 6-36 hours. In some embodiments the heating is carried out between about 12-36 hours. In some embodiments the heating is carried out between about 18-36 hours. In some embodiments the heating is carried out between about 24-36 hours.

In some embodiments the heating is carried out between about 0.1-24 hours. In some embodiments the heating is carried out between about 0.2-24 hours. In some embodiments the heating is carried out between about 0.3-24 hours. In some embodiments the heating is carried out between about 0.4-24 hours. In some embodiments the heating is carried out between about 0.5-24 hours. In some embodiments the heating is carried out between about 0.6-24 hours. In some embodiments the heating is carried out between about 0.7-24 hours. In some embodiments the heating is carried out between about 0.8-24 hours. In some embodiments the heating is carried out between about 0.9-24 hours. In some embodiments the heating is carried out between about 1-24 hours. In some embodiments the heating is carried out between about 3-24 hours. In some embodiments the heating is carried out between about 6-24 hours. In some embodiments the heating is carried out between about 12-24 hours. In some embodiments the heating is carried out between about 18-36 hours. In some embodiments the heating is carried out between about 18-24 hours.

In some embodiments the heating is carried at a temperature of about 105-140° C. and for at least 15 minutes, but no more than 72 hours and with the proviso that if the temperature is about 100° C., the heating is carried out for at least 24 hours.

In some embodiments the heating is carried at a temperature of about 105-140° C. and for at least 15 minutes, but no more than 72 hours and with the proviso that if the temperature is about 110° C., the heating is carried out for at least 30 minutes.

In some embodiments the heating is carried at a temperature of about 105-140° C. and for at least 15 minutes, but no more than 72 hours and with the proviso that if the temperature is about 120° C., the heating is carried out for not more than 24 hours.

In some embodiments the heating is carried at a temperature of about 105-140° C. and for at least 15 minutes, but no more than 72 hours and with the proviso that if the temperature is about 130° C., the heating is carried out for not more than 5 hours, such as no more than 3 hours, such as no more than 2 hours, such as no more than 1 hour.

In some embodiments the heating is carried at a temperature of about 105-140° C. and for at least 15 minutes, but no more than 72 hours and with the proviso that if the temperature is about 130° C., the heating is carried out for less than 1 hour.

In some embodiments, the heating is carried out in an oven and optionally under reduced pressure. The heating may be carried out in a tray oven.

In some embodiments there is provided a method of producing monosodium N-[8-(2-hydroxybenzoyl)-amino]caprylate (SNAC), the method comprising the following steps:
a. suspending or dissolving N-[8-(2-hydroxybenzoyl)-amino]caprylic acid in a solvent such as isopropanol;
b. adding a molar excess of a sodium containing salt such as sodium hydroxide as an aqueous solution to the suspension or solution from step (a) to form monosodium N-[8-(2-hydroxybenzoyl)-amino]caprylate;
c. isolating the so-formed monosodium N-[8-(2-hydroxybenzoyl)-amino]caprylate;
d. drying the monosodium N-[8-(2-hydroxybenzoyl)-amino]caprylate; and
e. heating the monosodium N-[8-(2-hydroxybenzoyl)-amino]caprylate at a temperature of above 90° C., such as at a temperature of about 105-140° C. for at least about 5 minutes, such as for at least about 15 minutes, and optionally for no more than 72 hours, such as not more than 24 hours.

### Step a.

N-[8-(2-hydroxybenzoyl)-amino]caprylic acid is dissolved or suspended in a suitable solvent. The suitable solvent may be an alcohol such as ethanol or isopropanol.

### Step b.

A sodium containing salt may be added to the solution or suspension obtainable in step a. The sodium containing salt may be sodium hydroxide. The sodium containing salt may be in the form of an aqueous solution or suspension obtainable in step a, such as a 10% aqueous solution or suspension, a 20% aqueous solution or suspension, a 30% aqueous solution or suspension, a 40% aqueous solution or suspension, a 50% aqueous solution or suspension, a 60% aqueous solution or suspension or a 70% aqueous solution or suspension. The sodium containing salt may be added in equimolar amounts to the solution or suspension obtainable in step a. The sodium containing salt may be added to the solution or suspension obtainable in step a in about 1.02 equivalents, in about 1.04 equivalents, in about 1.06 equivalent, in about 1.08 equivalent or in about 1.10 equivalents. The sodium containing salt may be added to the solution or suspension obtainable in step a at about 25° C., at about 30° C., at about 35° C., at about 40° C., at about 45° C. or at about 50° C.

After the addition is completed, the reaction mixture may be heated at, e.g., about 50° C., and cooled, e.g., to about 35° C., and can then be charged with seed crystal. After stirring at about 35° C. for about 1 hour, a suspension should form that can be slowly cooled to, e.g., about 30° C. and held at about 30° C. for about 1 hour to yield a thick suspension. Additional 2-propanol may be added at about 30° C., and the resulting slurry may then be cooled slowly to about 0° C. and aged for at least about 4 hours.

### Step c

The slurry comprising SNAC obtainable following step b, may be filtered and optionally washed. A mixture of isopropanol and water (about 10:1, v/v) may be used.

### Step d

The filtered and isolated SNAC obtainable following step c may be dried. SNAC may be dried under reduced pressure and/or at elevated temperature. For instance, SNAC may be dried in vacuum at about 70° C., at about 80° C. or at about 90° C. The temperature may be increased in a step-wise manner (e.g. going from the starting temperature to 60° C. to 70° C. to 90° C.) or in one step (e.g. going from the starting temperature to the desired temperature, e.g. 90° C., directly).

The drying step may be performed using an oven, a tray oven, a conical dryer, a spherical dryer, a biconical dryer or a fluidised bed to obtain SNAC polymorphic form A.

### Step e.

The dried SNAC polymorphic form A obtainable following step d may be heated.

In some embodiments, SNAC polymorphic form A obtainable following step d is heated at a temperature of about 105-135° C. In some embodiments SNAC polymorphic form A obtainable following step d is heated at a temperature of about 110-140° C., such as at a temperature of about 110-135° C., such as at a temperature of about 111-130° C. In some embodiments SNAC polymorphic form A obtainable following step d is heated at a temperature of about 115-124° C., such as at about 120° C. In some embodiments the heating is carried out at a temperature of about 111° C. or at a temperature of about 112° C. at a temperature of about 113° C. at a temperature of about 114° C. or at a temperature of about 115° C. or at a temperature of about 116° C. or at a temperature of about 117° C. or at a temperature of about 118° C. or at a temperature of about 119° C. or at a temperature of about 120° C. or at a temperature of about 121° C. or at a temperature of about 122° C. or at a temperature of about 123° C.

In some embodiments, SNAC polymorphic form A obtainable following step d is heated at a temperature of about 105-114° C., such as at about 110° C.

In some embodiments SNAC polymorphic form A obtainable following step d is heated at a temperature of about 115-124° C., such as at about 120° C.

In some embodiments SNAC polymorphic form A obtainable following step d is heated at a temperature of about 125-134° C., such as at about 130° C.

In some embodiments the heating of SNAC polymorphic form A obtainable following step d is carried out for at least 30 minutes or at least 45 minutes or at least 1 hour or at least 2 hours or at least 3 hours or at least 4 hours or at least 5 hours or at least 6 hours or at least 7 hours or at least 8 hours or at least 9 hours or at least 10 hours or at least 11 hours or at least 12 hours or at least 18 hours or at least 24 hours.

In some embodiments the heating of SNAC polymorphic form A obtainable following step d is carried out for less than 72 hours such a less than 66 hours, such as less than 60 hours such as less than 54 hours such as less than 48 hours such as less than 42 hours such as less than 36 hours such as less than 30 hours.

In some embodiments the heating of SNAC polymorphic form A obtainable following step d is carried out between about 15 minutes and 24 hours. In some embodiments the heating is carried out between about 20 minutes and 24 hours. In some embodiments the heating is carried out between about 30 minutes and 24 hours. In some embodiments the heating is carried out between about 45 minutes and 24 hours. In some embodiments the heating is carried out between about 1-24 hours. In some embodiments the heating is carried out between about 6-24 hours.

In some embodiments the heating of SNAC polymorphic form A obtainable following step d is carried out between about 1-72 hours. In some embodiments the heating is carried out between about 6-72 hours. In some embodiments the heating is carried out between about 12-72 hours. In some embodiments the heating is carried out between about 18-72 hours. In some embodiments the heating is carried out between about 24-72 hours.

In some embodiments the heating of SNAC polymorphic form A obtainable following step d is carried out between about 1-60 hours. In some embodiments the heating is carried out between about 6-60 hours. In some embodiments the heating is carried out between about 12-60 hours. In some embodiments the heating is carried out between about 18-60 hours. In some embodiments the heating is carried out between about 24-60 hours.

In some embodiments the heating of SNAC polymorphic form A obtainable following step d is carried out between about 1-54 hours. In some embodiments the heating is carried out between about 6-54 hours. In some embodiments the heating is carried out between about 12-54 hours. In some embodiments the heating is carried out between about 18-54 hours. In some embodiments the heating is carried out between about 24-54 hours.

In some embodiments the heating of SNAC polymorphic form A obtainable following step d is carried out between about 1-48 hours. In some embodiments the heating is carried out between about 6-48 hours. In some embodiments the heating of SNAC polymorphic form A obtainable following step d is carried out between about 12-48 hours. In some embodiments the heating is carried out between about 18-48 hours. In some embodiments the heating is carried out between about 24-48 hours.

In some embodiments the heating of SNAC polymorphic form A obtainable following step d is carried out between about 1-36 hours. In some embodiments the heating is carried out between about 6-36 hours. In some embodiments the heating is carried out between about 12-36 hours. In some embodiments the heating is carried out between about 18-36 hours. In some embodiments the heating is carried out between about 24-36 hours.

In some embodiments the heating of SNAC polymorphic form A obtainable following step d is carried out between about 0.1-24 hours. In some embodiments the heating of SNAC polymorphic form A obtainable following step d is carried out between about 0.2-24 hours. In some embodiments the heating of SNAC polymorphic form A obtainable following step d is carried out between about 0.3-24 hours. In some embodiments the heating of SNAC polymorphic form A obtainable following step d is carried out between about 0.4-24 hours. In some embodiments the heating of SNAC polymorphic form A obtainable following step d is carried out between about 0.5-24 hours. In some embodiments the heating of SNAC polymorphic form A obtainable following step d is carried out between about 0.6-24 hours. In some embodiments the heating of SNAC polymorphic form A obtainable following step d is carried out between about 0.7-24 hours. In some embodiments the heating of SNAC polymorphic form A obtainable following step d is carried out between about 0.8-24 hours. In some embodiments the heating of SNAC polymorphic form A obtainable following step d is carried out between about 0.9-24 hours. In some embodiments the heating is carried out between about 1-24 hours. In some embodiments the heating is carried out between about 3-24 hours. In some embodiments the heating is carried out between about 6-24 hours. In some embodiments the heating is carried out between about 12-24 hours. In some embodiments the heating is carried out between about 18-24 hours.

According to a second aspect of the invention, there is provided SNAC polymorphic form A exhibiting a mass increase of 1.5% or less when subjected to an increase in relative humidity from about 0% to about 65% RH at 25° C. as determined by DVS and/or comprising an XRDP peak at angles of diffraction 2Theta (2θ) of 8.7±0.2° measured using CuKa radiation having a FWHM of below 0.9° (2θ) such as below 0.85° (2θ).

In some embodiments, there is provided SNAC polymorphic form A exhibiting a mass increase of 1.3% or less, such as of 1.2% or less, such as of 1.1% or less, such as of 1.0% or less, when subjected to an increase in relative humidity from about 0% to about 65% RH at 25° C. as determined by DVS. In some embodiments, the SNAC polymorphic form A exhibiting a mass increase of 1.3% or less when subjected to an increase in relative humidity from about 0% to about 65% RH at 25° C. as determined by DVS has a representative X-ray powder diffraction pattern as provided in FIG. 1 or 2.

The invention relates in another aspect to SNAC polymorphic form A obtainable by a method according to the first aspect.

In some embodiments, SNAC polymorphic form A obtainable by a process according to the first aspect of the invention or a method according to a first alternative aspect of the invention, SNAC polymorphic form A exhibits a mass increase of 1.5% or less, such as of 1.3% or less, such as of 1.0% or less, when subjected to an increase in relative humidity from about 0% to about 65% RH at 25° C. as determined by DVS.

The invention in a third aspect relates to the use of SNAC polymorphic form A according to the second or alternative second aspect for the manufacture of a SNAC granule and/or a solid dosage form.

The invention also describes a process of manufacturing a pharmaceutical solid dosage form. In some embodiments, a process of manufacturing a solid pharmaceutical composition or dosage form comprises the steps of:
a. obtaining SNAC polymorphic form A according to the first aspect or alternative first aspect of the invention;
b. blending or mixing said SNAC polymorphic form A with a lubricant, such as magnesium stearate, and optionally with an active pharmaceutical ingredient such as a peptide, and optionally with one or more additional pharmaceutically acceptable excipients;
optionally c. granulating said blend or mixture obtainable in step b.
optionally d. mixing the granulates or granules obtainable from step c with additional excipients and
e. obtaining a solid pharmaceutical composition or dosage form such as a tablet.

In a fourth aspect, the invention relates to a solid or dry pharmaceutical composition suitable for oral administration comprising SNAC polymorphic form A. In some embodiments the solid pharmaceutical composition further comprises an active pharmaceutical ingredient and optionally at least one pharmaceutically acceptable excipient. The term “excipient” as used herein broadly refers to any components other than the active therapeutic ingredient(s) or active pharmaceutical ingredient(s) (API(s)). An excipient may be a pharmaceutically inert substance, an inactive substance, and/or a therapeutically or medicinally none-active substance. The excipients may serve various purposes, e.g. as a carrier, vehicle, filler, binder, lubricant, glidant, disintegrant, flow control agent, crystallization inhibitors, solubilizer, stabilizer, colouring agent, flavouring agent, surfactant, emulsifier or combinations of thereof and/or to improve administration, and/or absorption of the therapeutically active substance(s) or active pharmaceutical ingredient(s). The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013).

In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization inhibitors such as Povidone, etc.; solubilizers such as Pluronic, Povidone, etc.; colouring agents, including dyes and pigments such as iron oxide red or yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants. The composition may comprise a binder, such as povidone; starches; celluloses and derivatives thereof, such as microcrystalline cellulose, e.g., Avicel PH from FMC (Philadelphia, PA), hydroxypropyl cellulose hydroxylethyl cellulose and hydroxylpropylmethyl cellulose METHOCEL from Dow Chemical Corp. (Midland, MI); sucrose; dextrose; corn syrup; polysaccharides; and gelatine. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the composition comprises Avicel, such as Avicel PH 102. Suitable binders for wet granulation or dry granulation are corn starch, polyvinyl pyrrolidone (povidone), vinylpyrrolidone-vinylacetate copolymer (copovidone) and cellulose derivatives like hydroxymethylcellulose, hydroxyethylcellulose, hydroxypropylcellulose and hydroxylpropylmethylcellulose. In some embodiments the composition comprises povidone. In some embodiments the composition comprises a filler, which may be selected from lactose, mannitol, erythritol, sucrose, sorbitol, calcium phosphate, such as calciumhydrogen phosphate, microcrystalline cellulose, powdered cellulose, confectioner's sugar, compressible sugar, dextrates, dextrin and dextrose. In some embodiments the composition comprises microcrystalline cellulose, such as Avicel PH 102 or Avicel PH 200. In some embodiments the composition comprises a lubricant and/or a glidant. In some embodiments the composition comprises a lubricant and/or a glidant, such as talc, magnesium stearate, calcium stearate, zinc stearate, glyceryl behenate, glyceryl dibehenate, behenoyl polyoxyl-8 glycerides, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol etc. In some embodiments the composition comprises magnesium stearate or glyceryl dibehenate (such as the product Compritol® 888 ATO which consists of mono-, di- and triesters of behenic acid (C22) with the diester fraction being predominant). In some embodiments the composition comprises a disintegrant, such as sodium starch glycolate, polacrilin potassium, sodium starch glycolate, crospovidon, croscarmellose, sodium carboxymethylcellulose or dried corn starch. The composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants. As shown in the examples herein, the compositions of the invention have a very high content of the delivery agent. This very high content can be defined relative to the full content of the tablets including also the active pharmaceutical ingredient (e.g. a GLP-1 agonist) or alternatively relative to the total content of excipients excluding the active pharmaceutical ingredient. The description here below also refers to compositions consisting of specific ingredients, the active pharmaceutical ingredient and excipients, the term consisting is to be understood to nevertheless encompass trace amounts of any substance with no effect on the function of the composition, which may also be referred to as consisting essential of. Such substances can be impurities remaining in preparation of the active pharmaceutical ingredient or minimal amounts (below 1%) of any pharmaceutical acceptable excipient that do not affect the quality or absorption of the formulation.

In some embodiments, the active pharmaceutical ingredient is a GLP-1 agonist such as semaglutide.

In some embodiments, the pharmaceutical composition comprises a GLP-1 agonist and SNAC polymorphic form A according to the invention, wherein SNAC polymorphic form A is present in at least 60% w/w of the composition.

In further embodiments, SNAC polymorphic form A according to the invention constitutes above 71% w/w, such as above 72% w/w, such as above 73% w/w, such as above 74% w/w, such as above 75% w/w of said composition.

In further embodiments, SNAC polymorphic form A according to the invention constitutes above 81% w/w, such as above 82% w/w, such as above 83% w/w, such as above 84% w/w, such as above 85% w/w of said composition.

In further embodiments, SNAC polymorphic form A according to the invention constitutes above 91% w/w, such as above 92% w/w, such as above 93% w/w, such as above 94% w/w, such as above 95% w/w of said composition.

In some embodiments, the pharmaceutical composition comprises a GLP-1 agonist and SNAC polymorphic form A according to the invention, wherein SNAC polymorphic form A constitutes at least 90% w/w of the excipient of the composition.

### Definitions

As used herein, the term “about” or “approximately”, when used together with a numeric value (e.g. 5, 10%, ⅓), refers to a range of numeric values that can be less or more than the number. In some embodiments, the term “about” as used herein means+10% of the value referred to, and includes the value. For example, “about 5” refers to a range of numeric values that are 10%, 5%, 2%, or 1% less or more than 5, e.g. a range of 4.5 to 5.5, or 4.75 to 5.25, or 4.9 to 5.1, or 4.95 to 5.05.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The term “excipient” as used herein broadly refers to any component other than the active therapeutic ingredient(s) or active pharmaceutical ingredient(s) (API(s)).

The excipient may be a pharmaceutically inert substance, an inactive substance, and/or a therapeutically or medicinally non-active substance. The excipient may serve various purposes, e.g. as a carrier, vehicle, filler, binder, lubricant, glidant, disintegrant, flow control agent, crystallization inhibitors solubilizer, stabilizer, colouring agent, flavouring agent, surfactant, emulsifier or combinations of thereof and/or to improve administration, and/or absorption of the therapeutically active substance(s) or active pharmaceutical ingredient(s). The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013).

As used herein, “median particle size (D50)” refers to the particle size value where 50% of the particle sizes are smaller and 50% of the particle sizes are larger.

The terms “granulate” and “granules” are used interchangeably herein to refer to particles of composition material which may be prepared as described above.

The expression “heating is carried out at a temperature of” means that the heated SNAC polymorphic form A has the indicated temperature. Put differently, the temperature does not refer to the oven temperature but to the actual temperature of SNAC polymorphic form A. The temperature may for instance be controlled using a thermometer in the solid mass when heating SNAC polymorphic form A.

The term “crystal imperfections” is used to refer to interruptions by various defects in the regular periodic crystalline structure of the SNAC polymorphic form A, i.e. making an imperfection in the crystal structure. The interruption of the crystalline structure by these defects may cause a reduction of the crystallite size which therefore might impact the XRPD pattern by broadening the diffraction peaks. Representative XRPD patterns of SNAC polymorphic form A having increasing degrees of crystal imperfections are shown in FIGS. 1 to 3, respectively.

The term “polymorph” or “polymorphic form” refers to crystallographically distinct forms of a substance.

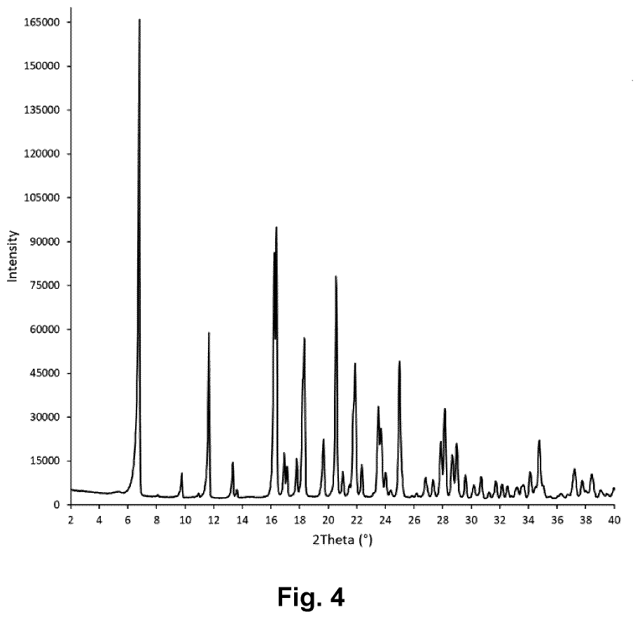

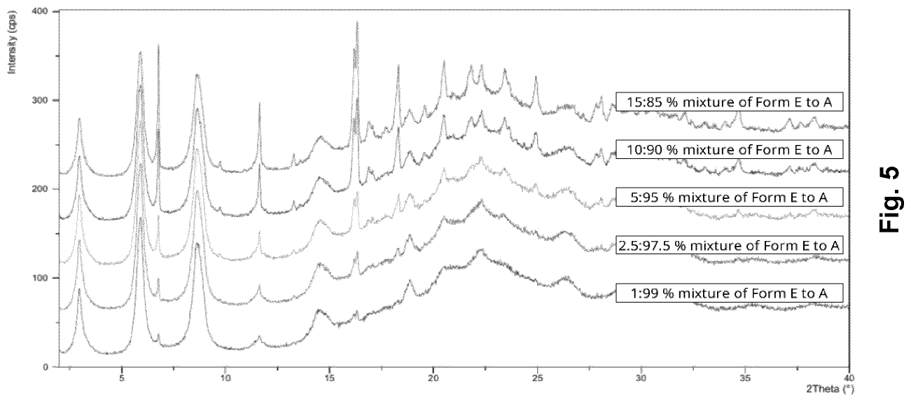

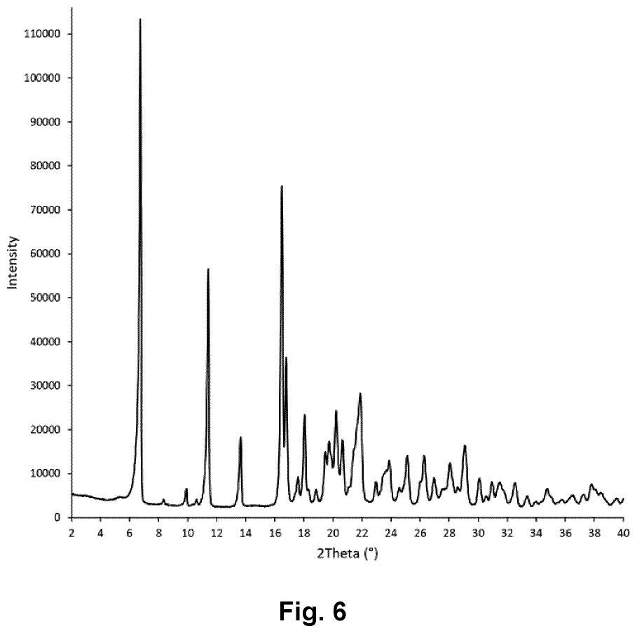

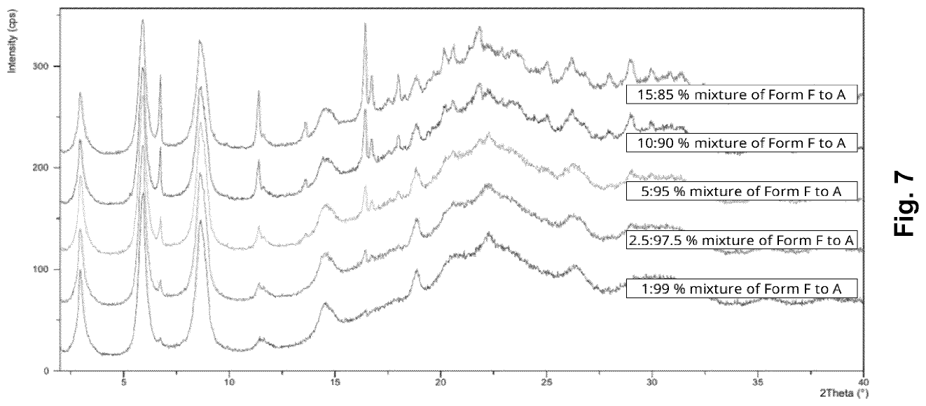

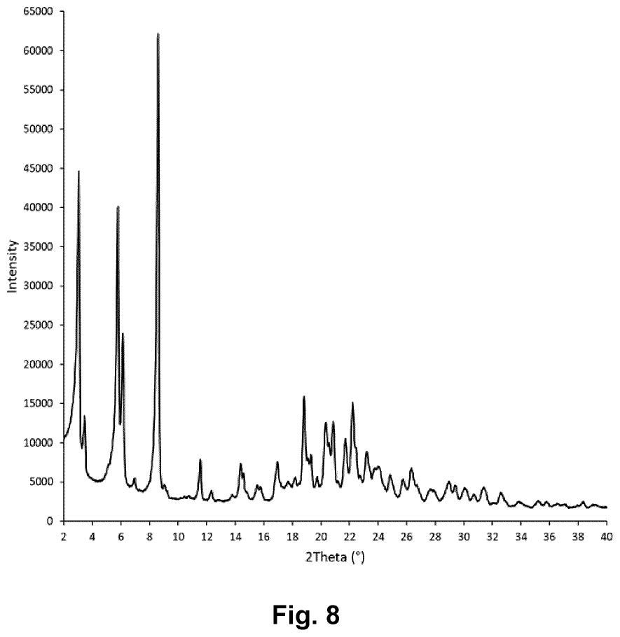

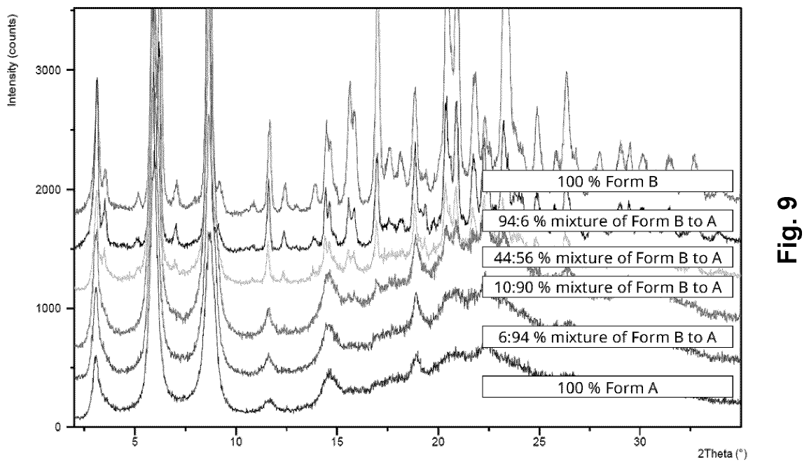

The term “polymorphic form A” refers to SNAC with the distinct periodic crystalline structure resulting in the XRPD pattern as shown in FIGS. 1 to 3. The presence of all six characteristic peaks at angles of diffraction 2Theta (2θ) of 3.0±0.2°, 6.0±0.2, 8.7±0.2°, 11.6±0.2°, 14.6±0.2°, and 18.9±0.2° differentiates the SNAC polymorphic form A from the other SNAC polymorphic forms such as E (see FIG. 4), F (see FIG. 6), and B (see FIG. 8) as demonstrated in FIGS. 5, 7, and 9, which shows mixtures of polymorphic form A with either E, F, or B. The term “polymorphic form A” also refers to SNAC with a melting point onset of 195-199° C. such as about 197° C. and without any other significant thermal events up until then and as determined by differential scanning calorimetry at scan speeds of up to 10° C./min and starting from room temperature.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### LIST OF EMBODIMENTS

1. A method of producing monosodium N-[8-(2-hydroxybenzoyl)-amino]caprylate exhibiting an X-ray powder diffraction patterns substantially as shown in FIG. 1 or FIG. 2, the method comprising the following steps:
a. suspending or dissolving N-[8-(2-hydroxybenzoyl)-amino]caprylic acid in a suitable solvent;
b. adding a sodium containing salt such as sodium hydroxide to form monosodium N-[8-(2-hydroxybenzoyl)-amino]caprylate;
c. isolating the so-formed monosodium N-[8-(2-hydroxybenzoyl)-amino]caprylate;
d. drying the monosodium N-[8-(2-hydroxybenzoyl)-amino]caprylate; and
e. heating, optionally under reduced pressure, the monosodium N-[8-(2-hydroxybenzoyl)-amino]caprylate at a temperature of above 90° C., such as at a temperature of about 105-140° C. for at least about 5 minutes, such as for at least about 15 minutes, and optionally for no more than 72 hours, such as no more than 24 hours.

2. A method of producing monosodium N-[8-(2-hydroxybenzoyl)-amino]caprylate exhibiting an X-ray powder diffraction pattern comprising peaks at angles of diffraction 2Theta (2θ) of 3.0±0.2°, 6.0±0.2, 8.7±0.2°, 11.6±0.2°, 14.6±0.2°, and 18.9±0.2° measured using CuKa radiation, the method comprising the following steps:
a. suspending or dissolving N-[8-(2-hydroxybenzoyl)-amino]caprylic acid in a suitable solvent;
b. adding a sodium containing salt such as sodium hydroxide form monosodium N-[8-(2-hydroxybenzoyl)-amino]caprylate;
c. isolating the so-formed monosodium N-[8-(2-hydroxybenzoyl)-amino]caprylate;
d. drying the monosodium N-[8-(2-hydroxybenzoyl)-amino]caprylate; and
e. heating, optionally under reduced pressure, the monosodium N-[8-(2-hydroxybenzoyl)-amino]caprylate at a temperature of a temperature of above 90° C., such as at a temperature of about 105-140° C. for at least about 5 minutes, such as for at least about 15 minutes.

3. A method of producing monosodium N-[8-(2-hydroxybenzoyl)-amino]caprylate polymorphic form A, the method comprising the following steps:
a. suspending or dissolving N-[8-(2-hydroxybenzoyl)-amino]caprylic acid in a suitable solvent;
b. adding a sodium containing salt such as sodium hydroxide form monosodium N-[8-(2-hydroxybenzoyl)-amino]caprylate;
c. isolating the so-formed monosodium N-[8-(2-hydroxybenzoyl)-amino]caprylate;
d. drying the monosodium N-[8-(2-hydroxybenzoyl)-amino]caprylate; and
e. heating, optionally under reduced pressure, the monosodium N-[8-(2-hydroxybenzoyl)-amino]caprylate at a temperature of above 90° C., such as at a temperature of about 105-140° C., for at least about 5 minutes, such as for at least about 15 minutes.

4. The method according to any one of the preceding embodiments, wherein the suitable solvent is an alcohol.

5. The method according to any one of the preceding embodiments, wherein the suitable solvent is isopropanol or ethanol.

6. The method according to any one of the preceding embodiments, wherein the suitable solvent is isopropanol.

7. The method according to any one of the preceding embodiments, wherein the sodium containing salt may be in form of an aqueous solution or suspension obtainable in step a, such as a 10% aqueous solution or suspension, a 20% aqueous solution or suspension, a 30% aqueous solution or suspension, a 40% aqueous solution or suspension, a 50% aqueous solution or suspension, a 60% aqueous solution or suspension or a 70% aqueous solution or suspension.

8. The method according to any one of the preceding embodiments, wherein the sodium containing salt may be added in equimolar amounts to the solution or suspension obtainable in step a.

9. The method according to any one of the preceding embodiments, wherein the sodium containing salt may be added in a molar excess to the solution or suspension obtainable in step a.

10. The method according to any one of the preceding embodiments, wherein the sodium containing salt may be added to the solution or suspension obtainable in step a in about 1.00 equivalents, in about 1.02 equivalents, in about 1.04 equivalents, in about 1.06 equivalent, in about 1.08 equivalent or in about 1.10 equivalents.

11. The method according to any one of the preceding embodiments, wherein the drying is performed using a vacuum dryer.

12. The method according to any one of the preceding embodiments, wherein the drying is performed using a biconical dryer, a conical dryer or a spherical dryer.

13. The method according to embodiment 11 or embodiment 12, wherein the vacuum dryer is an agitated vacuum dryer such a spherical dryer with agitators or a conical dryer with agitators.

14. The method according to any one of embodiments 11-14, wherein the vacuum dryer is a rotating dryer such as a tumble dryer.

15. A method of decreasing the amount of crystal imperfections in SNAC polymorphic form A, the method comprising the following steps:
A. providing SNAC polymorphic form A, and
B. heating, optionally under reduced pressure, the SNAC polymorphic form A provided in step as at a temperature of above 90° C., such as at a temperature of about 105-140° C. for at least about 5 minutes, such as for at least about 15 minutes, and optionally for no more than 72 hours such as no more than 24 hours.

16. A method of increasing the stability of SNAC polymorphic form A, the method comprising the following steps:
A. providing SNAC polymorphic form A, and
B. heating, optionally under reduced pressure, the SNAC polymorphic form A provided in step as at a temperature of above 90° C., such as at a temperature of about 105-140° C., for at least about 5 minutes, such as for at least about 15 minutes, and optionally for no more than 72 hours such as no more than 24 hours.

17. A method of reducing the hygroscopicity of monosodium N-[8-(2-hydroxybenzoyl)-amino]caprylate (SNAC) form A, the process comprising the following steps:
A. providing a SNAC polymorphic form A;
B. heating, optionally under reduced pressure, the SNAC polymorphic form A provided in step as at a temperature of above 90° C., such as at a temperature of about 105-140° C., for at least about 5 minutes, such as for at least about 15 minutes, and optionally for no more than 72 hours such as no more than 24 hours.

18. The method according to any one of embodiments 15-17, wherein SNAC polymorphic form A obtainable in step B exhibits an X-ray powder diffraction pattern substantially as shown in FIG. 1 or FIG. 2.

19. The method according to any one of embodiments 15-18, wherein SNAC polymorphic form A in step A. exhibits a mass increase of more than 1.3%, such as more than 1.4% when subjected to an increase in relative humidity from about 0% to about 65% relative humidity (RH) at 25° C. as determined by DVS.

20. The method according to any one of embodiments 15-19, wherein SNAC polymorphic form A in step A. exhibits a mass increase of more than 1.5% when subjected to an increase in relative humidity from about 0% to about 65% relative humidity (RH) at 25° C. as determined by DVS.

21. The method according to any one of embodiments 15-20, wherein SNAC polymorphic form A in step A. is characterised in that the peak at angles of diffraction 2Theta (2θ) of 8.7±0.2° measured using CuKa radiation has a FWHM of above 0.9° (2θ).

22. The method according to any one of the preceding embodiments, wherein said SNAC polymorphic form A is heated at a temperature of about 110-135° C.

23. The method according to any one of embodiments 1-21, wherein the heating is carried out at a temperature of about 105-114° C., such as at about 110° C.

24. The method according to any one of embodiments 1-21, wherein the heating is carried out at a temperature of about 115-124° C., such as at about 120° C.

25. The method according to any one of embodiments 1-21, wherein the heating is carried out at a temperature of about 125-134° C., such as at about 130° C.

26. The method according to any one of embodiments 1-11, wherein the heating is carried out at a temperature of about 111-135° C., at a temperature of about 118-135° C.

27. The method according to any one of embodiments 1-11 or embodiment 26, wherein the heating is carried out at a temperature of about 113-130° C., at a temperature of about 119-135° C.

28. The method according to any one of embodiments 1-27, wherein the heating is carried out for at least 20 minutes or at least 30 minutes or at least 45 minutes or at least 1 hour or at least 2 hours or at least 3 hours or at least 4 hours or at least 5 hours or at least 6 hours or at least 7 hours or at least 8 hours or at least 9 hours or at least 10 hours or at least 11 hours or at least 12 hours or at least 18 hours or at least 24 hours.

29. The method according to any one of embodiments 28, wherein the heating is carried out for less than 72 hours such a less than 66 hours, such as less than 60 hours such as less than 54 hours such as less than 48 hours such as less than 42 hours such as less than 36 hours such as less than 30 hours, such as less than 26 hours.

30. The method according to any one of embodiments 1-29, wherein the heating is carried out between about 0.1-24 hours, such as between about 0.2-24 hours.

31. The method according to any one of embodiments 1-30, wherein the heating is carried out between about 0.3-24 hours, such as between about 0.4-24 hours.

32. The method according to any one of embodiments 1-31, wherein the heating is carried out between about 0.5-24 hours, such as between about 0.6-24 hours.

33. The method according to any one of embodiments 1-32, wherein the heating is carried out between about 1-24 hours, such as between about 0.8-24 hours.

34. The method according to any one of embodiments 1-30, wherein the heating is carried out between about 3-72 hours.

35. The method according to any one of embodiments 1-30 or embodiment 34, wherein the heating is carried out between about 6-72 hours, such as between about 12-72 hours.

36. The method according to any one of embodiments 1-30 or embodiment 34, wherein the heating is carried out between about 12-72 hours.

37. The method according to any one of embodiments 1-30, wherein the heating is carried out between about 1-60 hours, such as between about 6-60 hours

38. The method according to any one of embodiments 1-30, wherein the heating is carried out between about 12-60 hours, the heating is carried out between about such as between about 18-60 hours, such as between about 24-60 hours.

39. The method according to any one of embodiments 1-27, wherein the heating is carried out between about 1-48 hours, such as between about 6-48 hours, such as between about 12-48 hours.

40. The method according to any one of embodiments 1-30, wherein the heating is carried out between about 18-48 hours, such as between about 24-48 hours.

41. The method according to any one of embodiments 1-30, wherein the heating is carried out between about 1-36 hours, such as between about 6-36 hours.

42. The method according to any one of embodiments 1-30, wherein the heating is carried out between about 12-36 hours, such as between about 18-36 hours.

43. The method according to any one of embodiments 1-33, wherein the heating is carried out between is carried out between about 24-36 hours.

44. The method according to any one of embodiments 1-30, wherein the heating is carried out between about 30 minutes to 24 hours, such as between about 1-24 hours.

45. The method according to any one of embodiments 1-30, wherein the heating is carried out between about 2-24 hours, such as between about 3-24 hours, such as between about 4-24 hours, such as between about 5-24 hours, such as between about 6-24 hours.

46. A SNAC polymorphic form A obtainable by the method or process according to any one of embodiments 1-45.

47. A SNAC polymorphic form A obtainable by the method or process according to any one of embodiments 1-45, wherein said SNAC polymorphic form A is characterised by exhibiting a mass increase of 1.5% or less, such as of 1.4% or less, when subjected to an increase in relative humidity from about 0% to about 65% relative humidity (RH) at 25° C. as determined by DVS.

48. A SNAC polymorphic form A obtainable by the method or process according to any one of embodiments 1-45, wherein said SNAC polymorphic form A is characterised by exhibiting a mass increase of 1.3% or less, such as of 1.2% or less, such as of 1.1% or less, such as of 1.0% or less, when subjected to an increase in relative humidity from about 0% to about 65% relative humidity (RH) at 25° C. as determined by DVS

49. A SNAC polymorphic form A obtainable by the method according to any one of embodiments 1-45, wherein the peak at angles of diffraction 2Theta (2θ) of 8.7±0.2° measured using CuKa radiation has a FWHM of below 0.9° (2θ), such as below 0.85° (2θ).

50. SNAC polymorphic form A according to embodiment 49, characterised in that the peak at angles of diffraction 2Theta (2θ) of 8.7±0.2° measured using CuKa radiation has a FWHM of between about 0.58-0.9° (2θ), such as between about 0.60-0.80° (2θ).

51. SNAC polymorphic form A according to embodiment 49 or embodiment 50, characterised in that the peak at angles of diffraction 2Theta (2θ) of 8.7±0.2° measured using CuKa radiation has a FWHM of between about 0.50-0.68° (2θ), such as between about 0.51-0.62° (2θ).

52. SNAC polymorphic form A according to embodiments 49-51, wherein the FWHM is measured by manual mode according to method 1.

53. SNAC polymorphic form A according to embodiments 49-51, wherein the FWHM is measured by automatic mode according to method 1.

54. A SNAC polymorphic form A exhibiting a mass increase of 1.5% or less when subjected to an increase in relative humidity from about 0% to about 65% relative humidity (RH) at 25° C. as determined by DVS.

55. The SNAC polymorphic form A according to embodiment 54 exhibiting a mass increase of 1.3% or less, such as of 1.2% or less, such as of 1.1% or less, when subjected to an increase in relative humidity from about 0% to about 65% relative humidity (RH) at 25° C. as determined by DVS.

56. The SNAC polymorphic form A according to embodiment 54 exhibiting a mass increase of 1.0% or less when subjected to an increase in relative humidity from about 0% to about 65% relative humidity (RH) at 25° C. as determined by DVS.

57. A SNAC polymorphic form A characterised in that the peak at angles of diffraction 2Theta (2θ) of 8.7±0.2° measured using CuKa radiation has a FWHM of below 0.9° (2θ), such as below 0.85° (2θ).

58. SNAC polymorphic form A according to embodiment 57, characterised in that the peak at angles of diffraction 2Theta (2θ) of 8.7±0.2° measured using CuKa radiation has a FWHM of between about 0.58-0.9° (2θ), such as between about 0.60-0.80° (2θ).

59. SNAC polymorphic form A according to embodiment 57 or embodiment 58, characterised in that the peak at angles of diffraction 2Theta (2θ) of 8.7±0.2° measured using CuKa radiation has a FWHM of between about 0.50-0.68° (2θ), such as between about 0.51-0.62° (2θ).

60. SNAC polymorphic form A according to embodiments 57-59, wherein the FWHM is measured by manual mode as described in method 1-X-ray powder diffraction.

61. SNAC polymorphic form A according to embodiments 57-59, wherein the FWHM is measured by automatic mode as described in method 1-X-ray powder diffraction.

62. A SNAC polymorphic form A comprising less than 99.5% crystal imperfections.

63. A SNAC polymorphic form A comprising less than 99% crystal imperfections, such as less than 98% crystal imperfection.

64. A SNAC polymorphic form A comprising less than 99% crystal imperfections, such as less than 97% crystal imperfection.

65. A SNAC polymorphic form A comprising less than 99% crystal imperfections, such as less than 96% crystal imperfection.

66. A SNAC polymorphic form A comprising less than 99% crystal imperfections, such as less than 95% crystal imperfection.

67. A SNAC polymorphic form A comprising between 0-5% crystal imperfection, such as between 0.01-5%, such as between 0.1-5%, such as between 0.5-5%. 68. Use of SNAC polymorphic form A obtainable by a method according to any one of embodiments 43-47 for the manufacture of a SNAC granule and/or a solid dosage form.

69. Use of SNAC polymorphic form A according to any one of embodiments 48-55 for the manufacture of a SNAC granule and/or a solid dosage form.

70. A solid pharmaceutical composition comprising SNAC polymorphic form A according to any one of the preceding embodiments.

71. The solid pharmaceutical composition according to embodiment 70, wherein SNAC polymorphic form A is in form of a powder or granulate.

72. The solid pharmaceutical composition according to embodiment 70 or embodiment 71, wherein SNAC polymorphic form A have a median particle size (D50) of between about 0.1-2000 μm.

73. The solid pharmaceutical composition according to embodiment 72, wherein the median particle size is between about 100-1000 μm, such as about 150-800 μm, such as about 200-600 μm.

74. The solid pharmaceutical composition according to embodiment 72, wherein the median particle size is between about 0.1-100 μm, such as about 0.5-80 μm, such as about 1-50 μm, such as about 5-30 μm.

75. The solid pharmaceutical composition according to any one of embodiments 70-75, further comprising an active pharmaceutical ingredient, a lubricant, and optionally one or more additional pharmaceutically acceptable excipients.

76. A process of manufacturing a solid pharmaceutical composition or dosage form comprising the steps of:
a. obtaining SNAC polymorphic form A according to the first aspect of the invention;
b. blending or mixing said SNAC polymorphic form A with a lubricant, such as magnesium stearate, and optionally with an active pharmaceutical ingredient such as a peptide, and optionally with one or more additional pharmaceutically acceptable excipients;
optionally c. granulating said blend or mixture obtainable in step b.
optionally d. mixing the granulates or granules obtainable from step c with additional excipients and
e. obtaining a solid pharmaceutical composition or dosage form such as a tablet.

77. SNAC polymorphic form A according to embodiment 55 exhibiting an X-ray powder diffraction patterns substantially as shown in FIG. 1 or FIG. 2.

### EXAMPLES

### Abbreviations

FWHM-full width at half maximum
XRPD-powder X-ray diffraction
DVS-dynamic vapour sorption
RH-relative humidity
SNAC-monosodium N-[8-(2-hydroxybenzoyl)-amino]caprylate

### General Methods of Detection and Characterisation

Monosodium N-[8-(2-hydroxybenzoyl)amino]caprylate (SNAC) can be prepared according to the procedure described in Example 2 of WO 2008/028859. Optionally a conical, biconical or spherical dryer may be used in the drying step instead of an oven as described in Example 2 of WO 2008/028859.

### Method 1-X-Ray Powder Diffraction

XRPD was performed using a Malvern Panalytical Empyrean diffractometer at ambient conditions. The diffraction pattern was measured at room temperature using an Empyrean Cu LFF HR (45 kV/40 mA) source and PIXcel3D-Medipix3 1×1 detector. The measurement was performed in the range of 2-40° 20 using a scan speed of 0.067335° 2θ/s and a step size of 0.0262606° 2θ. The samples were measured in transmission mode and with a spinner revolution time of 1 second. Approximately 200 mg of sample were placed between two sheets of Kapton Polyimide Thin-film. The measurements were performed using a Soller slit of 0.04 radians and a fixed divergence slit of 0.5° on the incident beam, and a 3 mm Anti scatter slit and a Soller slit of 0.04 radians in the diffracted beam.

The polymorphic form of the sample was determined by comparison of the diffractogram obtained for the sample with reference diffractograms for the SNAC polymorphs and solvates.

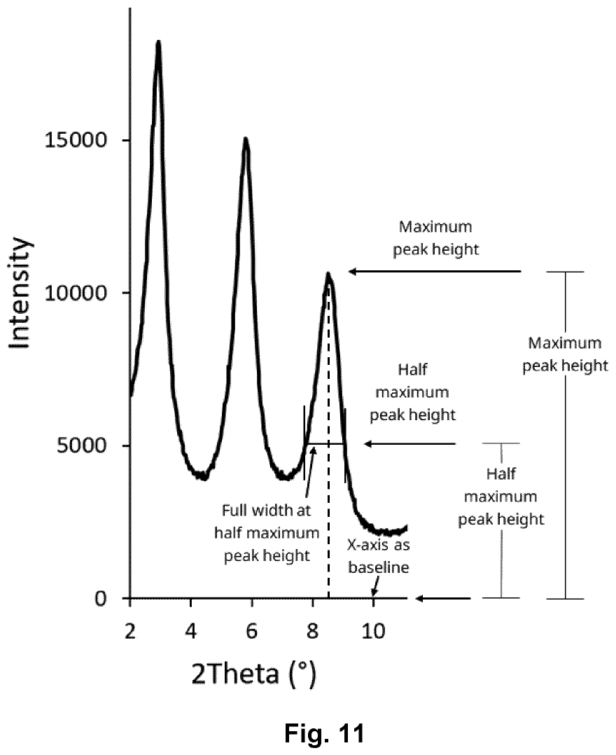

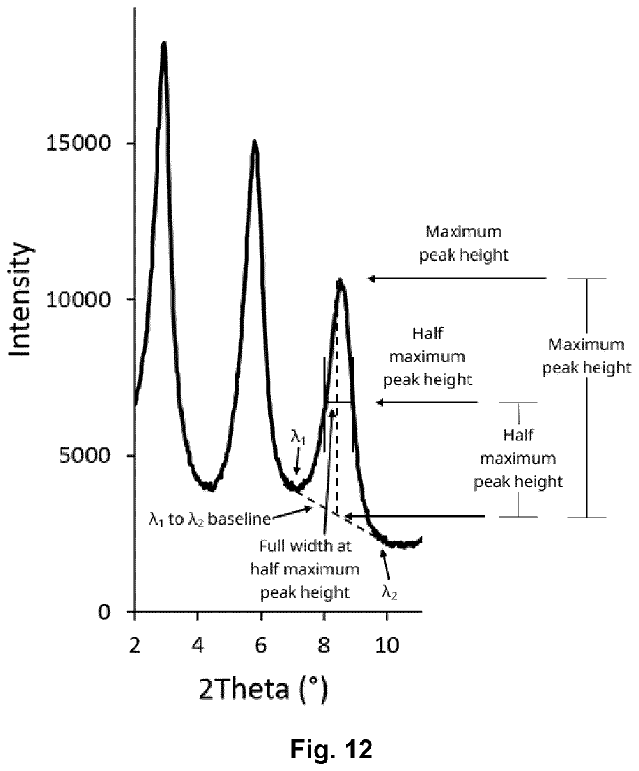

The degree of crystal imperfections was assessed by use of the FWHM as more crystal imperfections might cause peak broadening and lower peak height, which leads to higher FWHM values due to fewer lattice planes with identical orientation contributing to the diffraction peak. Oppositely, in the absence of crystal imperfections, lattice planes with identical orientation increase and result in narrower and higher diffraction peaks leading to smaller FWHM values. The FWHM was calculated using the characteristic peak at 8.7±0.2° 2θ by finding the apex of the peak and measuring the height of the peak applying the x-axis as the baseline (automatic mode) (see FIG. 11) or applying a baseline spanning the two surrounding troughs (manual mode) (see FIG. 12). This value is then divided in half to find the half height. Hereafter the width of the peak at that half height is measured and reported in ° 2θ. Alternatively, the peak at 6.0±0.2° 2θ may also be used to calculate the FWHM.

Method 2-X-Ray Powder Diffraction (XRPD) with Moisture Chamber

XRPD measurements at non-ambient conditions using an Anton Paar moisture chamber model MFD 2017, Type MCH-trans were performed using the same settings as described in method 1 for XRPD measurements. The moisture chamber allows for controlling the humidity and temperature of the samples during XRPD measurements.

### Method 3-Dynamic Vapor Sorption (DVS)

Moisture sorption/desorption data were measured by a Dynamic Vapour Sorption Advantage 1 from Surface Measurement Systems. Prior to analysis the samples were dried for up to about 48 h in desiccators containing phosphorus pentoxide. Approximately 20 mg of sample was placed into a balance pan and loaded into the instrument and the sample was then further dried for a minimum of 500 min in the instrument at 0% RH using a nitrogen purge. The resulting sample weight after the further drying was assigned as the 0% start weight and all weight gains and losses was calculated relative to this start weight. Sorption and desorption isotherms were collected over a range from 0 to 90% relative humidity (RH) using a nitrogen purge at 25° C. The threshold for equilibrium used for analysis was 0.002 to 0.0005 dm/dt.

### Example 1: Effect of Heating Temperature and Time

The purpose was to investigate the effect of heating temperature and time on the degree of crystal imperfections, on polymorphic forms, and on the hygroscopicity of SNAC polymorphic form A.

Samples from a batch of SNAC polymorphic form A (Batch 5) with a FWHM of 0.97° 2θ (automatic mode) and a moisture uptake of 1.98% at 65% RH from the beginning were heated to 90, 100, 110, 120, 130, and 140° C., respectively, in a tray oven. The samples were exposed to their respective temperatures for up to 72 h. The sample size was small enough to ensure that the samples equilibrated completely, within a few of minutes, to the oven temperature. Samples were taken during the 72 h of elevated temperature exposure and subjected to XRPD measurements in accordance with method 1 to determine the polymorphic form and impact on crystal imperfections using FWHM with the x-axis as the baseline (automatic mode). The results are shown in Tables 1 and 2.

TABLE 1
FWHM (automatic mode) (° 2θ) for the XRPD peak around 8.7 ± 0.2°
2θ for SNAC as a function of time at an elevated temperature.

| Temperature | Time at elevated temperature (h) | | | | | | | |
|---|---|---|---|---|---|---|---|---|
| (° C.) | 0 | 0.25 | 0.5 | 1 | 6 | 24 | 48 | 72 |
| 90 | 0.97 | 0.95 | 0.95 | 0.92 | 0.92 | 0.89 | 0.89 | 0.89 |
| 100 | 0.97 | 1.02 | 0.95 | 0.95 | 0.89 | 0.81 | 0.81 | 0.81 |
| 110 | 0.97 | 0.92 | 0.84 | 0.87 | 0.71 | 0.66 | 0.60 | 0.58 |
| 120 | 0.97 | 0.81 | 0.76 | 0.71 | 0.63 | 0.58 | 0.58 | 0.53 |
| 130 | 0.97 | 0.81 | 0.74 | 0.66 | 0.50 | 0.50 | 0.50 | 0.50 |
| 140 | 0.97 | 0.68 | 0.58 | 0.53 | 0.45 | 0.45 | 0.42 | 0.45 |

As shown in Table 1, the FWMH decreases when SNAC form A is exposed to elevated temperatures indicating a reduction in the degree of crystal imperfections as evident by the diffraction peak becoming narrower and higher. Furthermore, the degree of crystal imperfections is reduced the most with higher temperatures and longer exposure. The results shown in Table 1, show that the major reduction of crystal imperfections is obtained at temperatures from 90-140° C. after 6-24 h whereafter only a small further reduction is obtained. A marked part of the reduction in crystal imperfections is already achieved after around 1 h of exposure at the elevated temperature and already after 15 min a significant reduction is obtained.

TABLE 2
Polymorphic form of SNAC determined by XRPD as
a function of time at an elevated temperature.

| Temperature | Time at elevated temperature (h) | | | | | | | |
|---|---|---|---|---|---|---|---|---|
| (° C.) | 0 | 0.25 | 0.5 | 1 | 6 | 24 | 48 | 72 |
| 90 | A | A | A | A | A | A | A | A |
| 100 | A | A | A | A | A | A | A | A |
| 110 | A | A | A | A | A | A | A | A |
| 120 | A | A | A | A | A | A | A & B | A & B |
| 130 | A | A | A | A | A & B | A & B | A & B | A & B |
| 140 | A | A | A | A & B | A & B | A & B | A & B | A & B |

As shown in Table 2, SNAC polymorphic form A starts converting into polymorphic form B when exposed to temperatures starting from 120° C. As shown in Table 2, the higher the temperature, the earlier the onset time of conversion into polymorphic form B. The polymorphic form does not convert after exposure for up to 72 h at temperatures up to about 110° C. For example, at 120° C. conversion starts after more than 24 h, at 130° C. after 1 h and at 140° C. after 0.5 h.

Furthermore, samples were taken after 24 h of exposure at the indicated elevated temperatures and were subjected to dynamic vapor sorption measurements in accordance with method 3 to determine the hygroscopicity. The moisture uptake was measured at 65% RH. The results are shown in Table 3. The hygroscopicity is decreased as the moisture absorbed at 65% RH decreases when exposing SNAC polymorphic form A to elevated temperatures for 24 h.

TABLE 3
Moisture absorbed for SNAC at 65% RH (%) determined by
DVS as a function of time at an elevated temperature.

| | Time at elevated temperature (h) |
|---|---|
| Temperature (° C.) | 24 |
| 90 | 1.73 |
| 100 | 1.37 |
| 110 | 1.21 |
| 120 | 0.94 |
| 130 | 0.81 |
| 140 | 0.74 |

In conclusion, the degree of crystal imperfections and the hygroscopicity are both notably reduced for SNAC form A when exposing it to elevated temperatures above 90° C. for up to 72 h. At temperatures of 120° C. and above SNAC form A might start to convert into polymorphic form B if exposed for overly long time. A significant reduction in crystal imperfections is already achieved after 15 min and after 1 h then a significant part of the overall reduction is achieved and depending on the temperature then exposure times beyond 6 to 24 h only slightly reduces it further.

### Example 2-Effect of Heat Treatment on Different Batches of SNAC Polymorphic Form a

The purpose was to investigate the effect of heat treatment on the degree of crystal imperfections, on polymorphic forms, and on the hygroscopicity of several batches of SNAC polymorphic form A.

Samples from different batches of SNAC polymorphic form A were subjected to a heat treatment in a tray oven and the sample size was small enough to ensure that the samples equilibrated completely and within a couple of minutes to the oven temperature. Samples were taken from the batches before the heat treatment and after the heat treatment and subjected to XRPD measurements in accordance with method 1 to determine the polymorphic form and impact on crystal imperfections using FWHM with the x-axis as the baseline (auto mode). Furthermore, samples were also subjected to DVS measurements in accordance with method 3 to determine the impact of the heat treatment on the hygroscopicity.

TABLE 4
FWHM (automatic mode) (° 2θ) for the XRPD peak around 8.7 ± 0.2°
2θ, polymorphic form of SNAC, and moisture absorbed at 65% RH (%) for different
batches of SNAC polymorphic form A as a function of time at an elevated temperature.

| | | FWHM | | | | Moisture | |
|---|---|---|---|---|---|---|---|
| | | (automatic | | Polymorphic | | absorption at | |
| SNAC | Heat | mode) (° 2θ) | | form | | 65% RH (%) | |
| batch | treatment | Before | After | Before | After | Before | After |
| Batch 1 | 115° C. for 24 h | 1.08 | 0.60 | A | A | 2.66 | 1.26 |
| Batch 2 | 150° C. for 1 h | 1.21 | 0.42 | A | A & B | 2.90 | 1.04 |
| Batch 4 | 120° C. for 20 h | 0.79 | 0.58 | A | A | 0.83 | 0.84 |
| Batch 5 | 120° C. for 24 h | 0.97 | 0.58 | A | A | 1.98 | 0.94 |

The results from Table 4 show that crystal imperfections and hygroscopicity of SNAC polymorphic form A batches are reduced when subjected to an optimised heat treatment of 115° C. and above for up to 24 h. This is shown by the marked decrease in FWHM results making the diffraction peak narrower and higher due to less disorder and interruptions in the lattice planes and by the marked decrease in absorbed moisture at 65% RH making it much less hygroscopic. Furthermore, a heat treatment at 150° C. for up to 1 h results in form A starting to convert into form B and thus confirming the findings from example 1 that too high temperatures or overly long time at an elevated temperature might enable a conversion from polymorphic form A to form B.

In conclusion, a heat treatment at around 115 to 120° C. for up to 24 h of SNAC form A results in markedly reduced crystal imperfections and hygroscopicity. At temperatures above 130° C. then time exposure must be kept below 1 h to avoid conversion of polymorphic form A to polymorphic form B.

### Example 3: Relative Humidity Threshold for Onset of Conversion to SNAC Trihydrate (Polymorphic Form F)

The purpose was to evaluate the impact of the degree of crystal imperfections and the hygroscopicity on the required relative humidity for triggering conversion of SNAC form A to form F, which is a trihydrate form of SNAC.

Samples from different batches of SNAC polymorphic form A were subjected to increasing levels of relative humidity at ambient conditions inside a humidity chamber with the possibility of simultaneous determination of the polymorphic form in accordance with the XRPD method 2. The diffractograms were visually inspected for the appearance of characteristic diffraction peaks for the SNAC polymorphic form F in order to determine at which relative humidity polymorphic conversion of form A to F started.

TABLE 5
Relative humidity level required for onset of the conversion
of SNAC polymorphic form A to SNAC polymorphic form F.

| | | FWHM | | Moisture | Onset of |
|---|---|---|---|---|---|
| | | (manual | | absorption | trihydrate |
| | | mode) | Polymorphic | at 65% RH | conversion |
| SNAC batch | Heat treatment | (°2θ) | form | (%) | (% RH) |
| Batch 2 | None | 0.84 | A | 2.90 | 70 |
| Batch 3 | None | 0.66 | A | 1.95 | 75 |
| Batch 6 | None | 0.65 | A | 1.81 | 75 |
| Batch 7 | None | 0.61 | A | 2.06 | 70 |
| Batch 8 | None | 0.82 | A | 2.51 | 70 |
| Batch 8 | 115° C. for 24 h | 0.47 | A | 0.99 | 80 |
| Batch 9 | None | 0.60 | A | 1.29 | 80 |
| Batch 9 | 115° C. for 24 h | 0.52 | A | 0.98 | 80 |
| Batch X¹⁾ | None | 0.90 | A | 1.50 | — |

¹⁾“Batch X” refers to the sample described in example 1 of WO 2005/107462. The values were derived from example 1 and the figures disclosed in WO 2005/107462.

The result in Table 5 shows that the onset for polymorphic conversion of form A into form F occurs at 5-10% lower relative humidity when the FWHM results are higher. Likewise, it is found that the conversion into form F occurs at 5-10% lower relative humidity when the water absorption at 65% RH is higher.

In conclusion, a higher degree of crystal imperfections and increased hygroscopicity causes therefore a lower threshold for the start of form A to Form F conversion.

### Example 4: Effects of Increasing the Crystal Imperfections by Grinding

The purpose was to investigate the effect of a grinding induced increase in the degree of crystal imperfections in a batch of SNAC form A on the FWHM, the polymorphic form, and the moisture uptake.

A sample of about 1 g from a batch of SNAC form A were ground manually in a mortar with a pestle for about 10 min. Samples were taken before and after grinding, and subjected to XRPD measurements in accordance with method 1 to determine the polymorphic form and impact on crystal imperfections using FWHM with the x-axis as the baseline (auto mode). Furthermore, samples before and after grinding were also subjected to DVS measurements in accordance with method 3 to determine the impact of the heat treatment on the hygroscopicity.

TABLE 6
Effect of grinding SNAC polymorphic form A.

| | Ground in a | | | |
|---|---|---|---|---|
| SNAC | mortar with | FWHM (auto | Polymorphic | Moisture absorption |
| batch | pestle | mode) (°2θ) | form | at 60% RH (%) |
| Batch 10 | No | 0.79 | A | 1.19 |
| Batch 10 | Yes | 1.00 | A | 2.99 |

The results show that the moisture absorption at 65% RH increases extensively when the SNAC form A has been grinded manually in mortar and thus having more crystal imperfections.

In conclusion, the results show that the increased degree of crystal imperfections by grinding of SNAC form A results in markedly increased hygroscopicity as the moisture absorption is significantly higher after grinding.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Example 5: Crystal Imperfections for SNAC Manufactured According to Example 2 of WO2008/028859

The purpose was to investigate the effect of manufacturing SNAC according to the procedure described in example 2 of WO 2008/028859 on the degree of crystal imperfections, on polymorphic forms, and on the hygroscopicity of SNAC polymorphic form A.

SNAC polymorphic form A was manufactured according to the procedure described in example 2 of WO 2008/028859. Samples were taken after the drying at 90° C. for 18 h was completed and subjected to XRPD measurements in accordance with method 1 to determine the polymorphic form and impact on crystal imperfections using FWHM with the x-axis as the baseline (auto mode). Furthermore, a sample was also subjected to DVS measurements in accordance with method 3 to determine the hygroscopicity.

TABLE 7
Effecting of grinding SNAC polymorphic form A.

| | FWHM (auto | | Moisture absorption at |
|---|---|---|---|
| SNAC batch | mode) (°2θ) | Polymorphic form | 65% RH (%) |
| Batch 11 | 1.12 | A | 2.62 |

The results from Table 7 show that crystal imperfections and hygroscopicity of SNAC polymorphic form A batches are undesirably high when manufactured according to the procedure described in example 2 of WO2008/028859. The drying for 18 h at 90° C. is shown insufficient to reduce the crystal imperfections and hygroscopicity as also shown in example 1 with the drying at 90° C. for up to 72 h.

In conclusion, SNAC polymorphic form A manufactured according to the procedure described in example 2 of WO2008/02889 is undesirably high in crystal imperfections and hygroscopicity and would require a heat treatment above 90° C. to markedly reduce crystal imperfections and hygroscopicity.

1. A method for reducing the hygroscopicity of monosodium N-[8-(2-hydroxybenzoyl)-amino]caprylate (SNAC) form A, the process comprising:
a. providing a SNAC polymorphic form A; and
b. heating the SNAC polymorphic form A at a temperature of about 100-140° C. for at least 15 minutes.

2. The method according to claim 1, wherein the heating is carried out at a temperature of about 105-140° C. for a maximum of 72 hours.

3. The method according to claim 1, wherein the heating is carried out at a temperature of about 110-135° C.

4. The method according to claim 1, wherein the heating is carried out for at least 30 minutes.

5. The method according to claim 1, wherein the heating is carried out for at least 1 hour.

6. The method according to claim 1, wherein the heating is carried out for at least 6 hours.

7. The method according to claim 1, wherein the heating is carried out for not more than 30 hours, such as not more than 25 hours.

8. A monosodium N-[8-(2-hydroxybenzoyl)amino]caprylate (SNAC) polymorphic form A exhibiting an X-ray powder diffraction pattern comprising peaks at angles of diffraction 2Theta (2θ) of 2.94±0.06°, 5.82±0.05°, 8.6±0.1°, 11.45±0.15°, 14.4±0.2°, and 18.9±0.1° as measured using CuKa radiation, wherein the SNAC polymorphic form A exhibits a mass increase of 1.3% or less when subjected to an increase in relative humidity from about 0% to about 65% relative humidity (RH) at 25° C. as determined by dynamic vapour sorption (DVS) and/or wherein the peak at angles of diffraction 2Theta (2θ) of 8.7±0.2° as measured using CuKa radiation has a full width at half maximum (FWHM) of below 0.85° (2θ).

9. The SNAC polymorphic form A according to claim 8, wherein said SNAC polymorphic form A exhibits a mass increase of 1.1% or less when subjected to an increase in relative humidity from about 0% to about 65% relative humidity (RH) at 25° C. as determined by DVS.

10. The SNAC polymorphic form A according to claim 8, wherein the peak at angles of diffraction 2Theta (2θ) of 8.7±0.2° as measured using CuKa radiation has a FWHM of between about 0.58-0.90° (2θ).

11. The SNAC polymorphic form A according to claim 8, wherein the peak at angles of diffraction 2Theta (2θ) of 8.7±0.2° as measured using CuKa radiation has a FWHM of between about 0.50-0.68° (2θ).

12. The SNAC polymorphic form A according to claim 8, wherein the FWHM is measured by manual mode or by automatic mode.

13. (canceled)

14. A solid pharmaceutical composition comprising the SNAC polymorphic form A according to claim 8.

15. A process of manufacturing a solid pharmaceutical composition or dosage form comprising:
a. obtaining SNAC polymorphic form A according to claim 1;
b. blending or mixing said SNAC polymorphic form A with a lubricant, optionally with an active pharmaceutical ingredient, and optionally with one or more additional pharmaceutically acceptable excipients;
c. optionally granulating the blend or mixture obtained from step b.;
d. optionally mixing the granulates or granules obtained from step c with additional excipients; and
e. obtaining a solid pharmaceutical composition or dosage form.
